(19) United States
(12) Patent Application Publication
(10) Pub. No.: US 2022/0047528 A1
(43) Pub. Date: Feb. 17, 2022

(54) METHODS OF INHIBITING LYSINE-SPECIFIC DEMETHYLASE 1 (LSD-1)

(71) Applicant: Salarius Pharmaceuticals LLC, Houston, TX (US)

(72) Inventors: Shawn Blumberg, San Antonio, TX (US); Jonathan Bohmann, San Antonio, TX (US); Aundrietta D. Duncan, Houston, TX (US); Stephen Horrigan, Poolesville, MD (US); Bruce McCreedy, Raleigh, NC (US); Daniela Santiesteban, Houston, TX (US)

(21) Appl. No.: 17/385,508

(22) Filed: Jul. 26, 2021

Related U.S. Application Data
(60) Provisional application No. 63/056,109, filed on Jul. 24, 2020.

(51) Int. Cl. A61K 31/135; G01N 33/15

(52) U.S. Cl. CPC A61K 31/135; G01N 33/15

(57) ABSTRACT

Methods of inhibiting LSD1 activity by administering a compound that binds to SWIRM domain of LSD1 and inhibits LSD1 activity; methods of inhibiting LSD1 activity where the compound interacts with L547 residue within the hydrophobic pocket of LSD1; methods of determining the efficacy of a drug to be an LSD1 inhibitor wherein a candidate drug is screened to determine whether it binds to SWIRM domain of LSD1 and inhibits LSD1 activity.

### BACKGROUND

Epigenetic changes, which alter gene activity without altering DNA sequence, collaborate with genetic mistakes to promote cancer development and progression (Tsai, H. C. and Baylin, S. B. Cell Res 2011, 21 (3), 502-17; and Fullgrabe, J., Kavanagh, E., and Joseph, B. Oncogene 2011). The regulation of the modifications on DNA and the proteins associated with DNA has become an area of intense interest and the enzymes involved in these processes have been suggested as a new class of protein targets for drug development. The major proteins associated with DNA are histone proteins. Histone tails are subject to a variety of posttranslational modifications, such as phosphorylation, acetylation, methylation, and ubiquitination, and these modifications, especially acetylation and methylation on lysine residues, play a major role in the regulation of gene expression, and are often dysregulated in cancer (Fullgrabe, J., Kavanagh, E., and Joseph, B. Oncogene 2011).

Recently an enzyme called Lysine-Specific Demethylase 1 (LSD1) was found to catalyze the oxidative demethylation of monomethylated and dimethylated histone H3 at lysine 4 (H3K4me1 and H3K4me2) and lysine 9 (H3K9me1 and H3K9me2) through a flavin adenine dinucleotide (FAD)-dependent reaction (Shi, Y., et al. Cell 2004, 119 (7), 941-53; and Metzger, E., et al. Nature 2005, 437 (7057), 436-9), Whereas histone acetylation is associated with loose chromatin and gene activation, methylation of histones is less straightforward. Using the lysine residues regulated by LSD1 as an example, methylation at H3K4 is generally associated with gene activation, while methylation of H3K9 is associated with transcriptional repression.

LSD1 is an 852 amino acid protein, having the following sequence:

(SEQ ID NO: 1)
MLSGKKAAAA AAAAAAAATG TEAGPGTAGG SENGSEVAAQ
PAGLSGPAEV GPGAVGERTP RKKEPPRASP PGGLAEPPGS
AGPQAGPTVV PGSATPMETG IAETPEGRRT SRRKRAKVEY
REMDESLANL SEDEYYSEEE RNAKAEKEKK LPPPPPQAPP
EEENESEPEE PSGVEGAAFQ SRLPHDRMTS QEAACFPDII
SGPQQTQKVF LFIRNRTLQL WLDNPKIQLT FEATLQQLEA
PYNSDTVLVH RVHSYLERHG LINFGIYKRI KPLPTKKTGK
VIIIGSGVSG LAAARQLQSF GMDVTLLEAR DRVGGRVATF
RKGNYVADLG AMVVTGLGGN PMAVVSKQVN MELAKIKQKC
PLYEANGQAV PKEKDEMVEQ EFNRLLEATS YLSHQLDFNV
LNNKPVSLGQ ALEVVIQLQE KHVKDEQIEH WKKIVKTQEE
LKELLNKMVN LKEKIKELHQ QYKEASEVKP PRDITAEFLV
KSKHRDLTAL CKEYDELAET QGKLEEKLQE LEANPPSDVY
LSSRDRQILD WHFANLEFAN ATPLSTLSLK HWDQDDDFEF
TGSHLTVRNG YSCVPVALAE GLDIKLNTAV RQVRYTASGC
EVIAVNTRST SQTFIYKCDA VLCTLPLGVL KQQPPAVQFV
PPLPEWKTSA VQRMGFGNLN KVVLCFDRVF WDPSVNLFGH
VGSTTASRGE LFLFWNLYKA PILLALVAGE AAGIMENISD
DVIVGRCLAI LKGIFGSSAV PQPKETVVSR WRADPWARGS
YSYVAAGSSG NDYDLMAQPI TPGPSIPGAP QPIPRLFFAG
EHTIRNYPAT VHGALLSGLR EAGRIADQFL GAMYTLPRQA
TPGVPAQQSP SM.

It contains three domains: the SWIRM domain (amino acids 172-270), the AOL domains (amino acids 217-417 and 523-833), and the Tower domain (amino acids 418-522). (Chen, Y et al. Proceedings of the National Academy of Sciences 2006,103(38):13956-61). The contents of this publication are incorporated by reference in their entirety. There is currently one known mammalian homolog of LSD1 which is a protein variously designated LSD2, KDM1b, and AOF1. It shares a similar domain homology, but exhibits less than 31% sequence identity (Fang, R. et al. Molecular Cell 2010, 39:222-233). It has been shown that LSD2 is a H3K4me1/2 demethylase that specifically regulates histone H3K4 methylation within intragenic regions of its target genes (ibid.). Both LSD1 and LSD2 contain a SWIRM domain, a FAD coenzyme-binding motif, and a C-terminal amine oxidase domain, all of which are critical to the enzymatic activity. However, unlike LSD1, the protein LSD2 contains a CW-type zinc finger domain in its N-terminal domain, a region which is unstructured in LSD1. Furthermore, LSD2 lacks the “tower domain” of LSD1. At a cellular level, it has been suggested that LSD2 has a role in transcriptional regulation (ibid.). As expected, LSD2 appears to play a role in regulating DNA methylation as well, although the role in DNA methylation may be developmental stage specific (ibid.; Ciccone, D. N., et al. Nature 2009 461:415-418; Karytinos, A., et al. J. Biol. Chem. 2009 284:17775-17782; and Yang, Z., et al. Cell Res. 2010 20:276-287).

Several lines of evidence point to LSD1 as being a possible therapeutic target in cancer. LSD1 is reportedly over-expressed in a variety of tumors including neuroblastoma, ER-negative breast, bladder, lung, and colorectal tumors (Schulte, J. H., et al. Cancer Res 2009, 69 (5), 2065-71; Lim, S., et al. Carcinogenesis 2010, 31 (3), 512-20; and Hayami, S., et al. Int J Cancer 2011, 128 (3), 574-86). Increased methylation of the permissive H3K4 mark by LSD1 inhibition has been shown to reactivate expression of tumor suppressor genes in cancer models (Huang, Y., et al. Clin Cancer Res 2009, 15 (23), 7217-28). In addition, LSD1 has been found to associate with estrogen and androgen receptors leading to the specific demethylation of the repressive H3K9 mark, thereby increasing target gene expression (Metzger, E., et al. Nature 2005, 437 (7057), 436-9; and Garcia-Bassets, I., et al. Cell 2007, 128 (3), 505-18). Thus, depending upon cofactors bound to LSD1, demethylation by LSD1 can contribute to cancer through both the permissive H3K4 and the repressive H3K9 mark. Therefore, the inhibition of LSD1 might be an effective strategy for re-expression of epigenetically silenced tumor suppressor genes as well as down regulation of important cancer pathways in a number of cancer types.

SP-2577 compound (also known as seclidemstat) is a small-molecule LSD1 inhibitor. It is described in detail in U.S. Pat. Nos. 8,987,335 and 9,266,838, the contents of which are hereby incorporated by reference in their entirety. SP-2577 has the following structure:

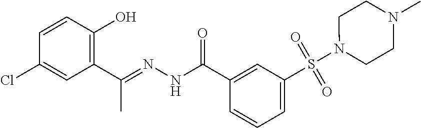 [SMILES: C/C(=N\NC(=O)c1cccc(S(=O)(=O)N2CCN(C)CC2)c1)c1cc(Cl)ccc1O]

### (E)-N′-(1-(5-chloro-2-hydroxyphenyl)ethylidene)-3-((4-methylpiperazin-1-yl)sulfonyl) benzohydrazide

While it has been known that SP-2577 inhibits LSD1 and has anti-cancer activity, up to the present invention it has not been known exactly how SP-2577 binds to LSD1. Accordingly, there is a need in the art to determine how SP-2577 may bind to LSD1 since this knowledge may be useful in determining the efficacy of other drugs to be an LSD1 inhibitor. This knowledge is also useful in arriving at methods of inhibiting LSD1 whereby a compound binds to the binding site.

### BRIEF SUMMARY OF THE INVENTION

In one aspect, the invention provides a method of inhibiting LSD1 activity comprising administering to a subject in need thereof a therapeutically effective amount of a compound that binds to SWIRM domain of LSD1 and inhibits LSD1 activity.

In a preferred embodiment, the compound is SP-2577.

In another preferred embodiment, the compound interacts with L547 residue within the hydrophobic pocket of LSD1.

In another preferred embodiment, the compound does not interact with W695 or T810 residues in the active site of LSD1.

In one aspect, the present invention provides methods and compositions for treating disease or disorder associated with an LSD activity dysfunction in a mammal comprising the step of administering to the mammal a therapeutically effective amount of a compound that binds to SWIRM domain of LSD1 and inhibits LSD1 activity.

Also disclosed are methods for inhibition of LSD activity in a mammal comprising the step of administering to the mammal a therapeutically effective amount of a compound that binds to SWIRM domain of LSD1 and inhibits LSD1 activity.

Also disclosed are methods for inhibiting LSD activity in at least one cell, comprising the step of contacting the at least one cell with an effective amount of a compound that binds to SWIRM domain of LSD1 and inhibits LSD1 activity.

In a further aspect, the invention relates to pharmaceutical compositions comprising a pharmaceutically acceptable carrier and an effective amount of a compound that binds to SWIRM domain of LSD1 and inhibits LSD1 activity.

In yet another embodiment, the invention provides methods of determining the efficacy of a drug to be an LSD1 inhibitor wherein a candidate drug is screened to determine whether the candidate drug binds to SWIRM domain of LSD1 and inhibits LSD1 activity, wherein such binding to SWIRM domain indicates that the candidate drug is an effective LSD1 inhibitor. In a preferred embodiment, the methods involve determining whether candidate drug compounds interact with W695 or T810 residues in the active site of LSD1. The absence of such interactions indicates that the candidate drug is an effective LSD1 inhibitor. In another preferred embodiment, the methods involve determining whether candidate drug compounds interact with L547 residue within the hydrophobic pocket. The presence of such interaction indicates that the candidate drug is an effective LSD1 inhibitor.

In yet another embodiment, the invention provides compounds suitable as LSD1 inhibitors, wherein such compounds are defined by conformation and structure such that these compounds bind to SWIRM domain of LSD1. In a preferred embodiment, such compounds do not interact with W695 or T810 residues in the active site of LSD1. In another preferred embodiment, such compounds interact with L547 residue within the hydrophobic pocket.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a three-dimensional image of in silico binding of SP-2577 and other LSD1 inhibitors to LSD1;

FIG. 2 is a three-dimensional image of in silico binding of a SP-2577 conformer to LSD1;

FIG. 3 is a three-dimensional image of in silico binding of another SP-2577 conformer to LSD1.

FIG. 4A is a correlation plot of 5 conformers per inhibitor molecule.

FIG. 4B is a correlation plot of 30 conformers per inhibitor molecule.

### DETAILED DESCRIPTION OF THE INVENTION

The present invention can be understood more readily by reference to the following detailed description of the invention and the Examples included therein.

Before the present compounds, compositions, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as ChemDraw™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “LSD” refers collectively to either or both LSD1 and LSD2.

As used herein, the terms “LSD1” and “lysine-specific demethylase 1” can be used interchangeably and refer to a histone demethylase encoded by the KDM1A gene. The KDM1A gene has a gene map locus of 1p36.12 as described by the Entrez Gene cytogenetic band, Ensembl cytogenetic band, and the HGNC cytogenetic band. The term LSD1 refers to a native protein that has 852 amino acids with a molecular weight of about 92903 Da, and is a member of the flavin monoamine oxidase family. The term LSD1 is inclusive of the protein, gene product and/or gene referred to by such alternative designations as: LSD1, KDM1; RP1-184J9.1; AOF2; BHC110; KIAA0601; LSD1; BRAF35-HDAC complex protein BHC110; FAD-binding protein BRAF35-HDAC complex, 110 kDa subunit; amine oxidase (flavin containing) domain 2; lysine-specific histone demethylase 1; lysine-specific histone demethylase 1A; flavin-containing amine oxidase domain-containing protein 2; lysine (K)-specific demethylase 1; amine oxidase (flavin containing) domain 2; and FAD-binding protein BRAF35-HDAC complex, 110 kDa subunit, as used by those skilled in the art.

As used herein, the terms “LSD2 and “lysine-specific demethylase 2 can be used interchangeably and refer to a histone demethylase encoded by the KDM1B gene. The KDM1B gene has a gene map locus of 6p22.3 as described by the Entrez Gene cytogenetic band, Ensembl cytogenetic band, and the HGNC cytogenetic band. The term LSD21 refers to a native protein that has 822 amino acids with a molecular weight of about 92098 Da, and is a member of the flavin monoamine oxidase family. The term LSD2 is inclusive of the protein, gene product and/or gene referred to by such alternative designations as: LSD2, AOF1; FLJ33898; FLJ34109; FLJ43328; C6orf193; DKFZp686I0412; OTTHUMP00000179125; bA204B7.3; dJ298J15.2; flavin-containing amine oxidase domain-containing protein 1; lysine-specific histone demethylase 2; lysine (K)-specific demethylase 1B; amine oxidase (flavin containing) domain 1; amine oxidase, flavin containing 1; lysine-specific histone demethylase 2; chromosome 6 open reading frame 193; and lysine-specific histone demethylase 1B, as used by those skilled in the art.

As used herein, the term “histone demethylase” refers to that group of enzymes which remove methyl groups from histone proteins. The term is inclusive of both histone lysine demethylases, i.e. enzymes which remove methyl groups from lysine residues in histones, and histone arginine demethylases, i.e. enzymes which remove methyl groups from arginine residues in histones.

As used herein, the term “histone lysine demethylase” or “lysine-specific histone demethylase” can be used interchangeably, and both refer to that group of enzymes which remove methyl groups from lysine residues of histone proteins. The histone lysine demethylases are a group of enzymes which comprise the following specific forms: LSD1, LSD2, JMJD2A, JMJD2B, JMJD2C and JMJD2D.

As used herein, the terms “optional” or “optionally” means that the subsequently described event or circumstance can or can not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation associated with a histone lysine demethylase dysfunction prior to the administering step. In some aspects of the disclosed method, the subject has been diagnosed with a need for inhibition of a histone lysine demethylase prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, zebra fish etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit a histone lysine demethylase. As a further example, “diagnosed with a need for inhibition of a histone demethylase” refers to having been subjected to a physical examination by a person of skill, for example, a physician, and found to have a condition characterized by a histone demethylase dysfunction. Such a diagnosis can be in reference to a disorder, such as a disorder of uncontrolled cellular proliferation, cancer and the like, as discussed herein. For example, the term “diagnosed with a need for inhibition of histone demethylase activity” refers to having been subjected to a physical examination by a person of skill, for example, a physician, and found to have a condition that can be diagnosed or treated by inhibition of histone demethylase activity. For example, “diagnosed with a need for treatment of one or more disorders of uncontrolled cellular proliferation associated with a histone demethylase dysfunction” means having been subjected to a physical examination by a person of skill, for example, a physician, and found to have one or more disorders of uncontrolled cellular proliferation associated with a histone demethylase dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a dysfunction of histone demethylase activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC₅₀ can refer to the concentration of a substance that is required for 50% agonism or activation in vivo, as further defined elsewhere herein. In a further aspect, EC₅₀ refers to the concentration of agonist or activator that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. For example, an IC₅₀ can refer to the concentration of a substance that is required for 50% inhibition in vivo or the inhibition is measured in vitro, as further defined elsewhere herein. Alternatively, IC₅₀ refers to the half maximal (50%) inhibitory concentration (IC) of a substance The inhibition can be measured in a cell-line such as AN3 CA, BT-20, BT-549, HCT 116, HER218, MCF7, MDA-MB-231, MDA-MB-235, MDA-MB-435S, MDA-MB-468, PANC-1, PC-3, SK-N-MC, T-47D, and U-87 MG. In a yet further aspect, the inhibition is measured in a cell-line, e.g. HEK-293 or HeLa, transfected with a mutant or wild-type mammalian histone demethylase, e.g. LSD1 or LSD2.

The term “pharmaceutically acceptable” describes a material that is not biologically or otherwise undesirable, i.e., without causing an unacceptable level of undesirable biological effects or interacting in a deleterious manner.

The term “stable,” as used herein, refers to compounds that are not substantially altered when subjected to conditions to allow for their production, detection, and, in certain aspects, their recovery, purification, and use for one or more of the purposes disclosed herein.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

### Binding Site of SP-2577 to LSD1

The structure and function of LSD1, as well as the SWIRM domain of LSD1 are well known in the art. The application incorporates by reference in its entirety the articles by Sehrawat et al, LSD1 activates a lethal prostate cancer gene network independently of its demethylase function, 10.1073/pnas.1719168115; and Tochio et al, Solution Structure of the SWIRM Domain of Human Histone Demethylase LSD1, Structure 14, 457-468, March 2006.

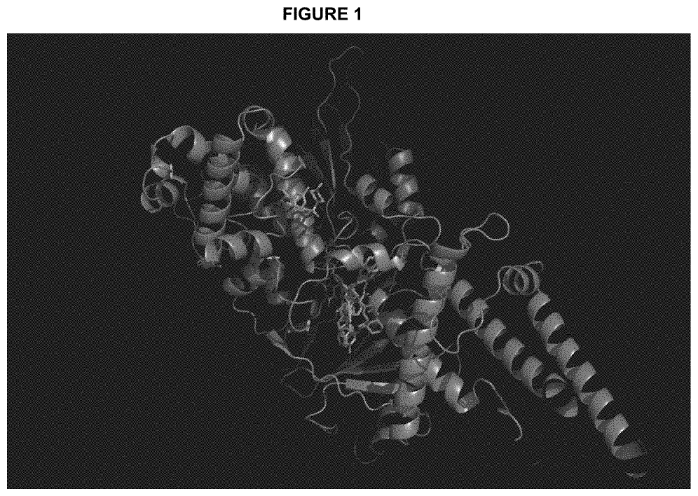

In silico binding of several known LSD1 inhibitors on the LSD1 protein was conducted to compare in silico binding characteristics. FIG. 1 shows a three-dimensional image of in silico binding of SP-2577 and other LSD1 inhibitors to LSD1. The purple color is LSD1: 5YJB—holo crystal structure; green is SP-2577; and magenta: other LSD1 inhibitors.

“Rhodium” a unique software program that enables prescreening of the three-dimensional structure of proteins and enzymes for pharmaceutical and biochemical research prior to drug synthesis. Rhodium software was internally developed at Southwest Research Institute (SwRI).

To simplify, in the Rhodium method, an inhibitor molecule 3D flexibility is represented by conformational sampling (5 to 30 internal states). The steps are as follows: a) generate search locations covering the surface of the protein (search grid); b) seed each search location with copies of an inhibitor conformer, generating trial candidate binding configurations (>10,000) at the different locations; and c) find binding modes by local optimization of seeds.

A crystal structure of LSD1 containing a non-covalent inhibitor was used for docking. Two binding site locations were observed with SP-2577, one of which had the shortest polar contact of all inhibitors in the study.

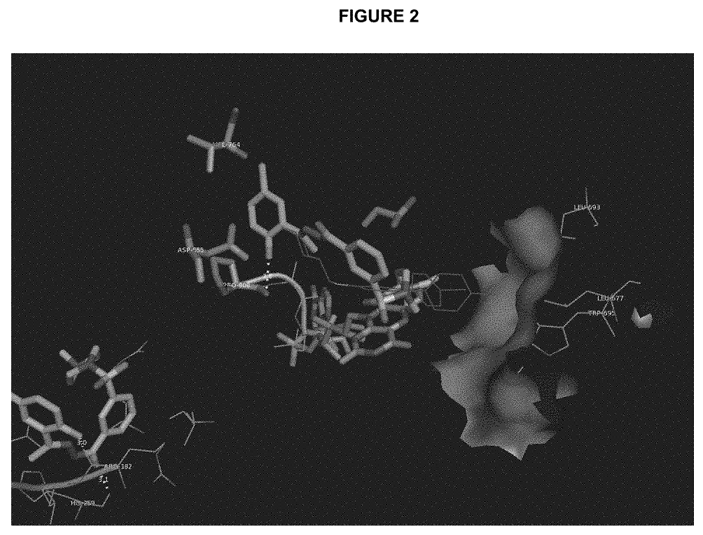

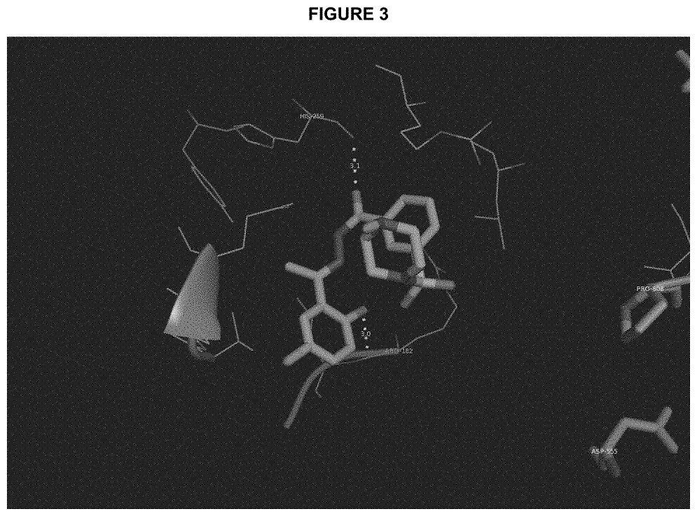

FIG. 2 and FIG. 3 show three-dimensional images of SP-2577 conformers docking to LSD1 obtained using Rhodium software. These Figures illustrate the binding of SP-2577 to LSD1.

The modeling has led to a surprising and unexpected discovery that SP-2577 binds to SWIRM domain of LSD1. Another surprising and unexpected discovery was that SP-2577 does not interact with W695 or T810 residues in the active side of LSD1 and instead interacts with L547 residue within the hydrophobic pocket of LSD1.

Thus, in one aspect, the invention provides a method of inhibiting LSD1 activity comprising administering to a subject in need thereof a therapeutically effective amount of a compound that binds to SWIRM domain of LSD1 and inhibits LSD1 activity.

In one embodiment, the compound is a small molecule compound.

In a preferred embodiment, the compound is SP-2577.

In another preferred embodiment, the compound interacts with L547 residue within the hydrophobic pocket of LSD1.

In another preferred embodiment, the compound does not interact with W695 or T810 residues in the active site of LSD1.

In one aspect, the present invention provides methods and compositions for treating disease or disorder associated with an LSD activity dysfunction in a mammal comprising the step of administering to the mammal a therapeutically effective amount of a compound that binds to SWIRM domain of LSD1.

Also disclosed are methods for inhibition of LSD activity in a mammal comprising the step of administering to the mammal a therapeutically effective amount of a compound that binds to SWIRM domain of LSD1.

Also disclosed are methods for inhibiting LSD activity in at least one cell, comprising the step of contacting the at least one cell with an effective amount of a compound that binds to SWIRM domain of LSD1.

In a further aspect, the invention relates to pharmaceutical compositions comprising a pharmaceutically acceptable carrier and an effective amount of a compound that binds to SWIRM domain of LSD1.

Examples of disorders associated with an LSD activity dysfunction include a disorder of uncontrolled cellular proliferation. In a yet further aspect, the disorder of uncontrolled cellular proliferation is cancer. In a yet further aspect, the cancer is a leukemia. In an even further aspect, the cancer is a sarcoma. In a still further aspect, the cancer is a solid tumor. In a yet further aspect, the cancer is a lymphoma.

It is understood that cancer refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

In various aspects, disorders associated with a histone demethylase dysfunction include neurodegenerative disorders. In a further aspect, the neurodegenerative disease is selected from Alzheimer's disease, Parkinson's disease, and Huntington's disease.

The compounds are further useful in a method for the prevention, treatment, control, amelioration, or reduction of risk of the diseases, disorders and conditions noted herein. The compounds are further useful in a method for the prevention, treatment, control, amelioration, or reduction of risk of the aforementioned diseases, disorders and conditions in combination with other agents.

The present invention is further directed to administration of the compounds of invention for improving treatment outcomes in the context of disorders of uncontrolled cellular proliferation, including cancer. That is, in one aspect, the invention relates to a co-therapeutic method comprising the step of administering to a mammal an effective amount and dosage of at least one compound of the invention in connection with cancer therapy.

In a further aspect, administration improves treatment outcomes in the context of cancer therapy. Administration in connection with cancer therapy can be continuous or intermittent. Administration need not be simultaneous with therapy and can be before, during, and/or after therapy. For example, cancer therapy can be provided within 1, 2, 3, 4, 5, 6, 7 days before or after administration of the compound. As a further example, cancer therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1;1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be co-administered, either in concomitant therapy or in a fixed combination.

In one aspect, the compound can be employed in combination with anti-cancer therapeutic agents or other known therapeutic agents.

In the treatment of conditions which require inhibition or negative modulation of LSD, an appropriate dosage level will generally be about 0.01 to 1000 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Additional Embodiments of the Invention

In yet another embodiment, the invention provides methods of determining the efficacy of a drug to be an LSD1 inhibitor wherein a candidate drug is screened to determine whether the candidate drug binds to SWIRM domain of LSD1 and inhibits LSD1 activity, wherein such binding to SWIRM domain indicates that the candidate drug is an effective LSD1 inhibitor. In a preferred embodiment, the methods involve determining whether candidate drug compounds interact with W695 or T810 residues in the active site of LSD1. The absence of such interactions indicates that the candidate drug is an effective LSD1 inhibitor. In another preferred embodiment, the methods involve determining whether candidate drug compounds interact with L547 residue within the hydrophobic pocket. The presence of such interaction indicates that the candidate drug is an effective LSD1 inhibitor.

In yet another embodiment, the invention provides compounds suitable as LSD1 inhibitors, wherein such compounds are defined by conformation and structure such that these compounds bind to SWIRM domain of LSD1. In a preferred embodiment, such compounds do not interact with W695 or T810 residues in the active site of LSD1. In another preferred embodiment, such compounds interact with L547 residue within the hydrophobic pocket.

The following Examples are intended to illustrate further certain embodiments of the invention and are not intended to limit the scope of the invention.

### EXAMPLES OF THE INVENTION

### Example 1. Rhodium Method Search of Seclidemstat's Binding Sites

Methods

“Rhodium” a unique software program that enables prescreening of the three-dimensional structure of proteins and enzymes for pharmaceutical and biochemical research prior to drug synthesis. Rhodium software was internally developed at Southwest Research Institute (SwRI). The SWRI Rhodium™ assisted design emphasizes three-dimensional space (sp3 carbons and chirality) to better fill the binding pocket and provide a means to better explore the chemical space. This software accelerates drug discovery by rapidly screening feasible binding sites and docking (computer simulated) poses that are not revealed in crystal structures. Rhodium can assist the analyst in these situations by ranking feasible poses of the ligand. Rhodium provides a docking score and a ranking of the binding locations for possible drugs during the screening process. The program uses a hierarchical scoring system to search the entire protein, allowing researchers to find novel target sites or multiple binding sites. The program generates a selection of ligand poses at each feasible site, which allows a researcher to extract the “top” pose for each site. This information is used to aid in the interpretation of experimental data, design new compounds or for biochemical engineering.

To simplify, in the Rhodium method, an inhibitor molecule 3D flexibility is represented by conformational sampling (5 to 30 internal states). The steps are as follows: a) generate search locations covering the surface of the protein (search grid); b) seed each search location with copies of an inhibitor conformer, generating trial candidate binding configurations (>10,000) at the different locations; and c) find binding modes by local optimization of seeds.

A crystal structure of LSD1 containing a non-covalent inhibitor was used for docking. Two binding site locations were observed with SP-2577, one of which had the shortest polar contact of all inhibitors in the study.

The experiment was performed to demonstrate a statistical correlation between a docking score called POP and potencies of certain known LSD inhibitors. The identical metric was subsequently used to characterize the subject compound. Each point corresponds to an inhibitor tested.

Results

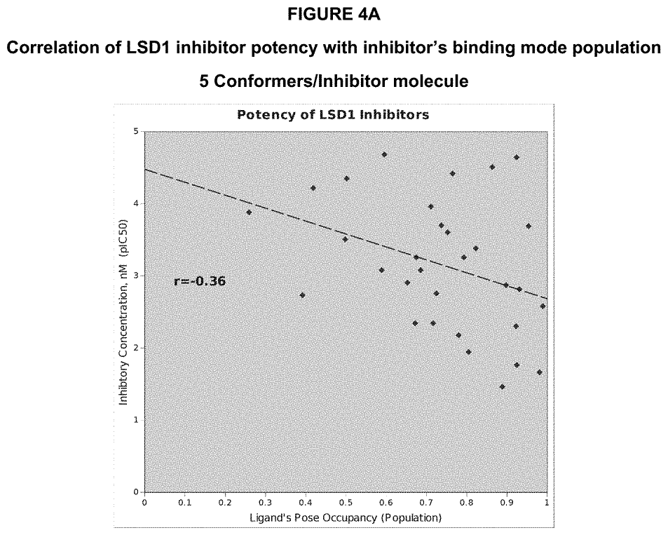

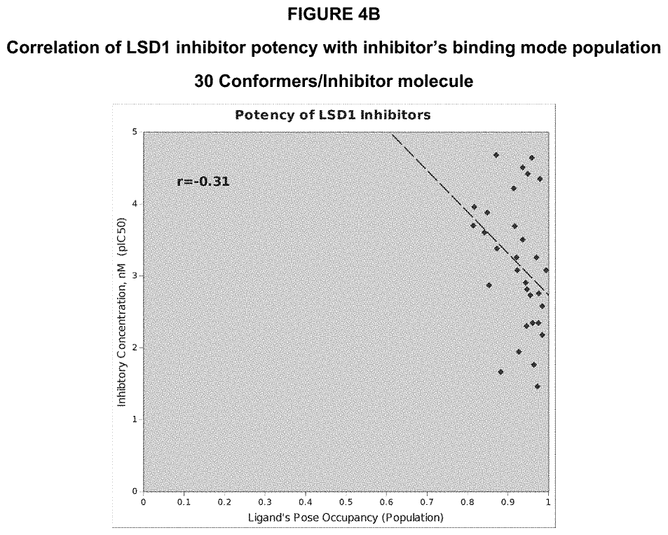

FIGS. 4A and 4B show two panels which demonstrate that a correlation is achieved even when the LSD inhibitors are represented in different 3D models—using 5 conformers per molecule (FIG. 4A) or 30 conformers per molecule (FIG. 4B). This means that the POP score is statistically suitable for comparing potency of the subject compound to others. The uncertainty can be determined from a separate analysis of the plots. Docking target is Protein Data Bank (PDB) 5YJB. The POP score is determined by a Boltzmann factor formula and represents the degree of localization of drug binding at a certain site on the protein surface. POP has a numerical range of 1.0 to 0.0. The observed POP numbers for seclidemstat (SP-2577) were: 0.908 for the primary site and 0.676 for the secondary site.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The ASCII text file “Sequence.txt” created on Oct. 6, 2021, having the size of 7,617 bytes, is incorporated by reference into the specification.

1. A method of inhibiting LSD1 activity comprising administering to a subject in need thereof a therapeutically effective amount of a compound that binds to SWIRM domain of LSD1 and inhibits LSD1 activity.

2. The method of claim 1, wherein the compound is a small molecule compound.

3. The method of claim 1, wherein the compound is SP-2577 which has the following structure 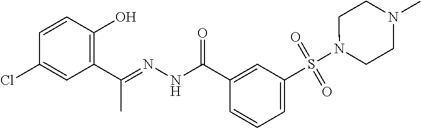 [SMILES: C/C(=N\NC(=O)c1cccc(S(=O)(=O)N2CCN(C)CC2)c1)c1cc(Cl)ccc1O]
or a pharmaceutically acceptable salt thereof.

4. The method of claim 1, wherein the compound interacts with L547 residue within the hydrophobic pocket of LSD1.

5. The method of claim 1, wherein the compound does not interact with W695 or T810 residues in the active site of LSD1.

6. A method for treating disease or disorder associated with an LSD activity dysfunction in a mammal comprising the step of administering to the mammal a therapeutically effective amount of a compound that binds to SWIRM domain of LSD1.

7. The method of claim 6, wherein the compound is a small molecule compound.

8. The method of claim 6, wherein the compound is SP-2577 which has the following structure 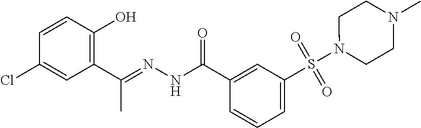 [SMILES: C/C(=N\NC(=O)c1cccc(S(=O)(=O)N2CCN(C)CC2)c1)c1cc(Cl)ccc1O]
or a pharmaceutically acceptable salt thereof.

9. The method of claim 6, wherein the compound interacts with L547 residue within the hydrophobic pocket of LSD1.

10. The method of claim 6, wherein the compound does not interact with W695 or T810 residues in the active site of LSD1.

11. The method of claim 6, wherein such disease is cancer.

12. A pharmaceutical composition comprising a pharmaceutically acceptable carrier and an effective amount of a compound that binds to SWIRM domain of LSD1 and inhibits LSD1 activity.

13. A method of determining the efficacy of a drug to be an LSD1 inhibitor wherein a candidate drug is screened to determine whether the candidate drug binds to SWIRM domain of LSD1 and inhibits LSD1 activity, wherein such binding to SWIRM domain indicates that the candidate drug is an effective LSD1 inhibitor.

14. The method of claim 13, wherein the method further comprises determining whether the candidate drug interacts with W695 or T810 residues of LSD1, wherein the absence of such interactions indicates that the candidate drug is an effective LSD1 inhibitor.

15. The method of claim 13, wherein the method further comprises determining whether the candidate drug interacts with L547 residue of LSD1 within the hydrophobic pocket of LSD1, wherein the presence of such interaction indicates that the candidate drug is an effective LSD1 inhibitor.

16. A compound suitable as an LSD1 inhibitor, wherein the compound binds to SWIRM domain of LSD1.


SEQUENCE LISTING (1 sequence)

SEQ ID NO: 1 - protein, 852 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM